(19) United States
(12) Patent Application Publication
(10) Pub. No.: US 2007/0083103 A1
(43) Pub. Date: Apr. 12, 2007

(54) Arrangement for acquiring an object

(76) Inventors: Jan Boese, Eckental (DE); Martin Kleen, Furth (DE); Andreas Meyer, Mohrendorf (DE); Marcus Pfister, Bubenreuth (DE); Norbert Rahn, Forchheim (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/529,681

(22) Filed: Sep. 28, 2006

(30) Foreign Application Priority Data
Sep. 28, 2005 (DE) 10 2005 046 416.5

(51) Int. Cl. A61B 5/05

(52) U.S. Cl. 600/407

(57) ABSTRACT

The invention relates to an arrangement having a 3D device, the 3D device being embodied for acquiring an objects and generating a 3D acquisition result representing the object at least partially in at least three dimensions. The arrangement also has a 2D device, the 2D device being embodied for acquiring the object and generating a 2D acquisition result representing the object in at least two dimensions. The 2D acquisition result represents the object at least partially, in particular a top view of the object, a view through the object or a section through the objects. The invention is characterized in that the 3D devices and the 2D devices are connected to one another, mechanically electrically, in such a way that a part of the 3D acquisition result corresponding to an object location can be assigned to a part of the 2D acquisition result corresponding to the same object location.

### CROSS REFERENCE TO RELATED APPLICATIONS

This application claims priority of German application No. 10 2005 046 416.5 filed Sep. 28, 2005, which is incorporated by reference herein in its entirety.

### FIELD OF THE INVENTION

The invention relates to an arrangement having a 3D device, the 3D device being embodied for acquiring an object and generating a 3D acquisition result representing the object at least partially in at least three dimensions. The arrangement also has a 2D device, the 2D device being embodied for acquiring the object and generating a 2D acquisition result representing the object in at least two dimensions. The 2D acquisition result represents the object at least partially, in particular a top view of the object, a view through the object or a section through the object.

### BACKGROUND OF THE INVENTION

3D devices in the form of computed tomography systems, magnetic resonance tomography systems, positron emission computed tomography systems or single-photon emission computed tomography systems are known from the prior art. Such 3D devices can record an object, a patient for example, in 3 spatial dimensions. A user-selectable sectional or through-view image which is required for example for an intervention procedure can then be selected from the acquisition result.

With 3D devices known from the prior art, the process of acquisition and in particular a subsequent evaluation by a user, a physician for example, currently takes up a lot of time to the extent that an acquisition and evaluation of this kind is regularly performed prior to an intervention or in critical phases of the intervention following time-consuming repositioning of the patient or for monitoring after the intervention. During the intervention 2D acquisition results, generated for example by means of a C-arm X-ray device, must be mentally reconciled by a user, a physician for example, with the acquisition results of the 3D device in order to compare the 2D acquisition result with the 3D acquisition result.

### SUMMARY OF THE INVENTION

The problem underlying the invention is therefore that the 3D acquisition result generated by a 3D device and the 2D acquisition result generated by a 2D device, a C-arm X-ray system for example, and obtained for example during an intervention are difficult to compare with each other in order, for example, to relocate an organ, a vessel or the like represented in each case by an acquisition result.

The aforementioned problem is solved by an arrangement of the type cited in the introduction, wherein the 3D device and the 2D device are in each case connected to each other, in particular mechanically, in such a way that a part of the first acquisition result corresponding to an object location can be assigned to a part of the second acquisition result corresponding to the same object location.

A 3D acquisition result can be a 3D dataset which represents an object at least partially in at least three dimensions. For example, a 3D dataset can represent an object in at least 3 spatial dimensions. A 4D dataset can represent an object in 3 spatial and in a further time-dependent dimension. In the case of a 4D dataset the object has therefore been acquired in addition as a function of time.

A 2D acquisition result can be a 2D dataset which represents the object at least partially. For example, the 2D dataset can represent a top view of the object, a view through the object or a section through the object. In another exemplary embodiment a 3D dataset can represent an object in at least three dimensions, with two dimensions being location-dependent and therefore spatial, and one dimension being time-related and therefore time-dependent.

A 3D dataset can also preferably contain data corresponding to a plurality of voxel object points and the voxel object points together at least partially represent the object in at least three dimensions, with one voxel object point representing one location in an object.

A 2D dataset can contain data corresponding to a plurality of pixels of an image of an object, the pixels together at least partially representing the image of an object.

An object can be represented at least partially in that a part of the object, an organ or a vessel, for example, in the case of a patient, is represented. Alternatively or in addition thereto, partially representing an object can be realized by a spatial distancing of acquisition points that are adjacent to one another.

A mechanical connection of the 2D device to the 3D device can be, for example, a rigid connection between a housing part of the 2D device and a housing part of the 3D device. Alternatively thereto, a detachable rigid connection can also be provided between the aforementioned housing parts.

In a preferred embodiment, the arrangement can, for example, have a receiving apparatus with a receiving surface for receiving an object, in particular a patient, that can be used jointly by the 3D device and the 2D device. The receiving apparatus is embodied to supply the object either to the 3D device for the purpose of being acquired by the 3D device or to the 2D device for the purpose of being acquired by the 2D device. The receiving apparatus can also preferably be embodied to connect the 3D device mechanically to the 2D device. In this embodiment the receiving apparatus advantageously forms a connecting piece which is disposed between the 3D device and the 2D device.

In an alternative embodiment the 3D device and the 2D device can in each case be connected to a base, for example a floor or a junction plate base, which can form a rigid connecting piece. The receiving apparatus can be part of the 3D device or the 2D device.

The receiving apparatus is preferably embodied to move the receiving surface to a predetermined first position in the acquisition range of the 3D device, or optionally to a predetermined second position in the acquisition range of the 2D device.

A calibration of the arrangement can advantageously be performed at these positions that are known relative to one another.

By means of the receiving apparatus an object can advantageously be supplied to, in each case, predetermined positions in the acquisition ranges of the aforementioned devices, with the result that parts of the acquisition result corresponding to an object location can be assigned to one another.

In a preferred embodiment the receiving apparatus is embodied to generate a calibration signal at at least one predetermined position of the receiving surface. This advantageously enables acquisition locations which in each case represent the same object location to be assigned in a simple manner.

The receiving apparatus is preferably embodied to supply the receiving surface to the acquisition range of the 3D device or optionally the acquisition range of the 2D device by translational movement and/or rotational movement.

The receiving apparatus is also preferably embodied to swivel the receiving surface about at least one spatial axis. As a result an object which is connected, in particular rigidly, to the receiving surface can advantageously be acquired by the 2D device at an acquisition angle which corresponds to an acquisition angle of an acquisition by the 3D device.

The receiving apparatus can also preferably be embodied to swivel the receiving surface about two or three spatial axes which are in particular orthogonal to one another.

In a preferred embodiment the 2D device is electrically connected to the 3D device.

The arrangement preferably has a coordinate memory which is connected in each case to the 3D device and the 2D device. The 2D device and the 3D device are each embodied to generate an object coordinates dataset corresponding to an object location and to store the object coordinates dataset in the coordinate memory. This advantageously enables a calibration of the arrangement to be performed.

The arrangement also preferably has an assignment unit with an input for a calibration signal, the assignment unit being embodied to assign a 3D object coordinates dataset generated by the assignment unit to the 2D object coordinates dataset generated by the 2D device as a function of a calibration signal received on the input side.

This advantageously further simplifies a calibration of the arrangement.

In an advantageous embodiment variant the arrangement has a magnetic field navigator which is embodied to generate a magnetic field with a spatial orientation. The spatial orientation of the magnetic field can be changed as a function of a user interaction in such a way that a magnetizable or magnetized object, in particular a distal catheter end of a catheter, can be orientated in an effective range of the magnetic field correspondingly spatially to said field.

The magnetic field navigator is preferably at least indirectly connected to the coordinate memory and embodied to read out an object coordinates dataset stored in the coordinate memory and to output a position of the magnetizable or magnetized object relative to the read out object coordinates dataset.

Alternatively to this embodiment, the magnetic field navigator can generate the object location of the magnetizable or magnetized object in the form of coordinates which correspond to those given by the 2D object coordinates dataset or the 3D object coordinates dataset.

The magnetic field navigator advantageously enables an end of a catheter to be moved to a position which corresponds to a predetermined position which is represented by a 3D acquisition result.

A magnetic field navigator can advantageously be a magnetic field navigator of the company Stereotaxis.

In an advantageous embodiment the arrangement can have a position sensor which is embodied to detect the position of a location-indicating object—for example a catheter end. The position sensor is preferably at least indirectly connected to the coordinate memory and embodied to read out an object coordinates dataset stored in the coordinate memory and output a position of the location-indicating object relative to the read out object coordinates dataset.

Alternatively to this embodiment the position sensor can generate the object location of the location-indicating object in the form of coordinates which correspond to those given by the 2D object coordinates dataset or the 3D object coordinates dataset.

A position sensor can advantageously be a position sensor of the company Biosense Webster.

In a preferred embodiment the arrangement has an image display unit which is connected at least indirectly to the assignment unit. The arrangement is embodied to display jointly, spatially and/or on a time-dependent basis, the object represented in each case by the 2D acquisition result and by the 3D acquisition result on at least one image display unit.

The invention also relates to a method for acquiring an object, preferably by means of an arrangement of the aforementioned type.

The method comprises the following steps:
at least partial acquisition of an object in at least three dimensions and generation of a 3D acquisition result representing the object at least partially in at least three dimensions,
generation of a 3D object coordinates dataset corresponding to an object location,
storing of the 3D object coordinates dataset,
at least partial acquisition of an object in at least two dimensions and generation of a 2D acquisition result representing the object in at least two dimensions, the 2D acquisition result representing the object at least partially, in particular a top view of the object, a view through the object or a section through the object,
generation of a 2D object coordinates dataset corresponding to an object location, storing of the 2D object coordinates dataset,
assignment of the 3D object coordinates dataset to the 2D object coordinates dataset as a function of a calibration signal.

A 2D object coordinates dataset can represent an object location in two or three spatial dimensions.

In a further preferred embodiment the method additionally has the following step:
joint display, in particular joint display spatially or on a time-dependent basis or both of the object represented in each case by the 2D acquisition result and the 3D acquisition result on at least one image display unit.

A spatially joint display can be a representation in a common space or in a common plane. Object parts that are different relative to one another can also be displayed in a common space or in a common plane.

### BRIEF DESCRIPTION OF THE DRAWINGS

The invention will now be explained below with reference to figures, in which:

FIG. 1 shows in schematic form an exemplary embodiment of an arrangement having a 3D device, a 2D device and a receiving surface, and

FIG. 2 shows in schematic form an exemplary embodiment of an arrangement having a 3D device and a 2D device and a magnetic field navigator.

### DETAILED DESCRIPTION OF THE INVENTION

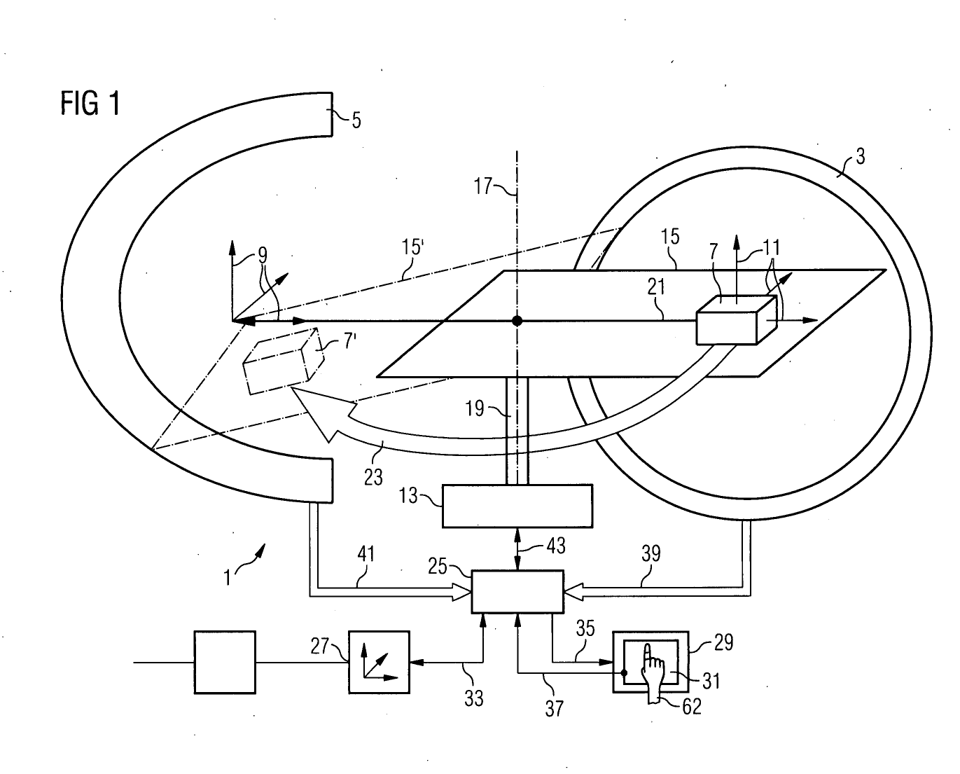

FIG. 1 shows in schematic form an arrangement 1 having a 3D device 3, a 2D device 5 and a receiving apparatus 13.

The 3D device 3, for example a SPECT scanner (SPECT=Single-Photon Emission Computed Tomography) is embodied to acquire an object 7 and generate a 3D acquisition result representing the object 7 at least partially in three dimensions. The 3D device can also be embodied to generate a 3D object coordinates dataset corresponding to an acquisition location and to output said dataset on the output side.

The 3D acquisition result can be a 3D dataset which is formed by a plurality of voxel image points which together at least partially represent the object 7.

The 3D dataset can also contain the object coordinates dataset which represents the acquisition location of the object 7 acquired by the 3D device.

The 2D device, for example a C-arm X-ray device, is embodied for acquiring the object and generating a 2D acquisition result representing the object in at least two dimensions. The 2D acquisition result at least partially represents the object, in particular a top view of the object, a view through the object or a section through the object.

The receiving apparatus 13 has a receiving surface 15 and a swiveling connection 19. The receiving surface 15 is connected to the receiving apparatus 13 via the swiveling connection 19. The receiving apparatus 13 is embodied to swivel the receiving surface 15 about a swiveling axis 17 as a function of a user interaction signal received on the input side. The receiving surface 15 is shown in a swiveling position 15′.

The receiving surface 15 is embodied to receive an object 7, a patient for example.

In the embodiment shown in FIG. 1, the receiving apparatus 13 is embodied to swivel the receiving surface 15 about a further swiveling axis (not shown in this diagram). The further swiveling axis is arranged vertically relative to the swiveling axis 17. The further swiveling axis causes the receiving surface 15 to run in the swiveling position 15′ in a plane which is inclined relative to the plane which is described by the receiving surface 15 in the swiveled-back position.

For example, the receiving apparatus 13 can swivel the receiving surface 15 back and forth in the range of a swiveling angle of 180 degrees.

The arrangement 1 also has an assignment device 25 which is connected to the receiving apparatus 13 via a bidirectional connecting cable 43. The assignment device 25 is connected to the 2D device 5 via a data bus 41 and to the 3D device 3 via a data bus 39.

The arrangement 1 also has a coordinate memory 27 which is connected to the assignment device 25 via a connecting cable 33.

The arrangement 1 also has an image display unit 29. The image display unit 29 has a touch-sensitive surface 31, the touch-sensitive surface 31 being connected to the assignment unit 25 via a connecting cable 37, and the image display unit 29 being connected to the assignment unit 25 via a connecting cable 35. The image display unit 29 can be, for example, a TFT display (TFT=Thin Film Transistor).

The touch-sensitive surface 31 is embodied to generate a touch signal as a function of a touching of the touch-sensitive surface 31, which touch signal corresponds to a touch location of the touch-sensitive surface 31, and to output said signal via the connecting cable 37 on the output side. Also shown is a hand of a user 62 which can generate a touch signal indirectly by touching the touch-sensitive surface 31.

The principle of operation of the arrangement 1 will now be explained as follows:

The 3D device 3 can send the generated 3D object coordinates dataset via the data bus 39 to the assignment device 25.

Also shown are object coordinates 11 which represent the acquisition location of the object 7 at which the object 7 was acquired by the 3D device.

The assignment device 25 is embodied to output the object coordinates dataset received over the data bus 39 on the output side via the connecting cable 33 and store it in the coordinate memory 27.

The receiving apparatus 13 can generate a calibration signal as a function of a swiveling position of the receiving surface 15. The receiving apparatus 13 can now generate a calibration signal which corresponds to the swiveling position of the receiving surface 15 in the acquisition range of the 3D device, and to send said calibration signal via the connecting cable 43 to the assignment unit 25. Said assignment unit 25 can send the object coordinates dataset representing an acquisition location and received via the data bus 39 to the coordinate memory 27 via the connecting cable 33 as a function of the calibration signal received via the connecting cable 43 and store it there.

The receiving apparatus 13 can now swivel the receiving surface 15 into the swiveling position 15′—for example as a function of a touch signal generated by the touch-sensitive surface 31—and thereby move the object 7 located on the receiving surface 15 along the swiveling direction 23 into the object position 7′ and therefore into the acquisition range of the 2D device 5. A resulting movement of the object 7 is represented by the movement direction arrow 21.

The 2D device 5, for example a C-arm X-ray device, is embodied to acquire an object and generate a 2D acquisition result representing the object in at least two dimensions. In this embodiment the 2D acquisition result represents, for example, a view through the object 7. The 2D device is embodied to output the 2D acquisition result, for example a 2D dataset which has a plurality of pixel image points which together represent the view through the object 7, via the data bus 41 on the output side.

The receiving apparatus 13 can now generate a calibration signal corresponding to the swiveling position of the receiving surface 15′ and send said signal via the connecting cable 43 to the assignment unit 25. The 2D device is embodied to generate a 2D object coordinates dataset corresponding to an acquisition location of the object in the swiveling position 7′ and to send said dataset via the data bus 41 to the assignment unit 25 on the output side.

The assignment unit 25 can send the 2D object coordinates dataset received via the data bus 41 as a function of the calibration signal received via the connecting cable 43 and representing the swiveling position 15′ via the connecting cable 33 to the coordinate memory 27 and store it there.

With the object coordinates datasets stored in the coordinate memory 27, a 2D acquisition result, represented by a 2D dataset, can now be assigned by the assignment unit 25 to a 3D acquisition result, represented by a 3D dataset. On the basis of the object coordinates datasets stored in the coordinate memory 27, the assignment unit 25 can thus assign components of the 2D dataset and the 3D dataset corresponding to precisely one object location to one another and generate a corresponding assignment result.

The assignment unit 25 can output the assignment result on the output side and send it via the connecting cable 35 to the image display unit 29 for joint display on the image display unit 29.

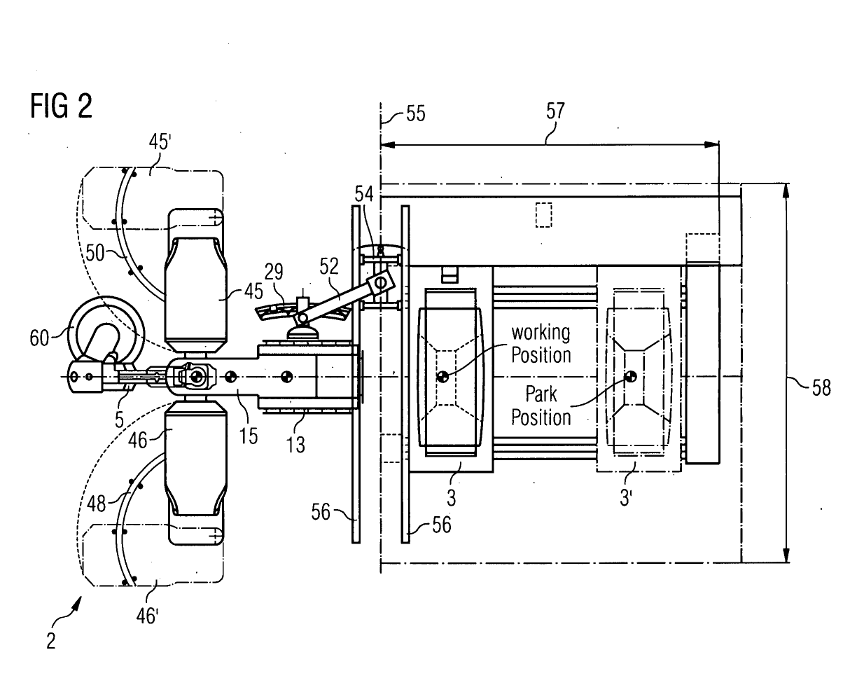

FIG. 2 shows an exemplary embodiment of an arrangement 2 having a 3D device 3 already described in FIG. 1,
a 2D device 5 already described in FIG. 1,
a receiving apparatus 13 having a receiving surface 15 which have likewise already been described in FIG. 1.

The arrangement 2 also shows an image display unit 29 which has likewise already been described in FIG. 1 and in this exemplary embodiment in arrangement 2 is connected to a carriage 54 via a swiveling arm 52. The carriage 54 is embodied to be moved back and forth on rails 56 along a longitudinal axis 55.

In this exemplary embodiment the 2D device is a C-arm X-ray device with a pedestal 60.

The receiving apparatus 13 can swivel an object located on the receiving surface 15, for example a patient, optionally into the acquisition range of the 2D device 5 or into the acquisition range of the 3D device 3.

The 3D device 3 is shown in an acquisition position. Also shown is a park position 3′ of the 3D device.

In addition to the arrangement 1 shown in FIG. 1, the arrangement 2 has a magnetic field navigator. The magnetic field navigator has a magnetic field head 46 and a magnetic field head 45. The magnetic field head 46 is swivel-mounted and can be swiveled on a slide rail 48 into the swiveling position 46′. The magnetic field head 45 is swivel-mounted and can be swiveled on a slide rail 48 into the swiveling position 45′.

The magnetic field heads 45 and 46 are each embodied to generate a magnetic field with a spatial orientation. The magnetic field navigator can change the spatial orientation of the magnetic field as a function of a user interaction signal, for example a touch signal generated by the touch-sensitive surface 31 in FIG. 1.

The magnetic field navigator can be connected to the coordinate memory 27 shown in FIG. 1 and is embodied to read out object coordinates datasets stored in the coordinate memory 27 and to output an object location of a magnetizable or magnetized object which is located in the aligned magnetic field relative to the read out object coordinates dataset.

The magnetic field navigator can send said dataset, which represents the object location of the magnetizable object, to the assignment unit 25 shown in FIG. 1 for displaying on the image display unit 29 shown in FIG. 1.

Also shown are the spacing dimension 58, which measures 500 centimeters, and the spacing dimension 57, which measures 455 centimeters.

1-5. (canceled)

6. A medical arrangement, comprising:
a 3D device which generates a 3D acquisition result corresponding to an object location at least partially representing the object in at least three dimensions and a 3D object coordinate dataset corresponding to a 3D acquisition location;
a 2D device which generates a 2D acquisition result corresponding to the same object location at least partially representing the object in at least two dimensions and a 2D object coordinate dataset corresponding to a 2D acquisition location;
a receiving apparatus comprising a receiving surface for receiving the object which:
moves the receiving surface back and forth from a first predetermined position in an acquisition range of the 3D device to a second predetermined position in an acquisition range of the 2D device by swiveling the receiving surface about an axis,
generates a calibration signal at at least one of the first and second predetermined positions of the receiving surface; and
an assignment unit connected to the 3D device, the 2D device and the receiving apparatus which assigns the 3D object coordinate dataset to the 2D object coordinate dataset as a function of the calibration signal.

7. The medical arrangement as claimed in claim 6, further comprising a coordinate memory connected to the 3D device and the 2D device which stores the 3D and 2D object coordinate datasets.

8. The medical arrangement as claimed in claim 7, wherein a portion of the 3D acquisition result is assigned to a portion of the 2D acquisition result based on the 3D and 2D object coordinate datasets stored in the coordinate memory.

9. The medical arrangement as claimed in claim 8, further comprising a display unit which displays the assignment result.

10. The medical arrangement as claimed in claim 7, further comprising a magnetic field navigator connected to the coordinate memory which generates a magnetic field with a spatial orientation as a function of a user interaction such that a magnetizable or permanently magnetic object is orientated in an effective range of the magnetic field.

11. The medical arrangement as claimed in claim 10, wherein the magnetic field navigator:
reads out the 3D or 2D object coordinate dataset stored in the coordinate memory, and
outputs a position of the magnetizable or permanently magnetic object relative to the read out 3D or 2D object coordinate dataset.

12. The medical arrangement as claimed in claim 11, wherein the position of the magnetizable or permanently magnetic object is in an area of a distal end of a catheter.

13. The medical arrangement as claimed in claim 6, wherein the 3D device is mechanically or electrically connected to the 2D device.

14. The medical arrangement as claimed in claim 6, wherein the 3D device is a 3D image device and the 2D device is a 2D image device.

15. The medical arrangement as claimed in claim 6, wherein the 2D acquisition result is a top view of the object, or a view through the object or a section through the object.

16. The medical arrangement as claimed in claim 6, wherein the receiving apparatus supplies the object by a translational or rotational movement of the receiving surface.

17. The medical arrangement as claimed in claim 6, wherein the object is a live patient.

18. A method for acquiring an object in a medical procedure, comprising:
acquiring the object by a 3D device in a first predetermined position in an acquisition range of the 3D device;
generating a 3D acquisition result representing the object at least partially in at least three dimensions;
generating a 3D object coordinate dataset corresponding to a 3D object location;
calculating a first calibration signal corresponding to the first predetermined position;
storing the 3D object coordinate dataset;
acquiring the object by a 2D device in a second predetermined position in an acquisition range of the 2D device;
generating a 2D acquisition result representing the object at least partially in at least two dimensions;
generating a 2D object coordinate dataset corresponding to a 2D object location;
storing the 2D object coordinate dataset; and
assigning the 3D object coordinate dataset to the 2D object coordinate dataset as a function of the first calibration signal.

19. The method as claimed in claim 18, wherein a second calibration signal is calculated corresponding to the second predetermined position and the 3D object coordinate dataset is assigned to the 2D object coordinate dataset as a function of the second calibration signal.

20. The method as claimed in claim 18, further comprising jointly displaying the object represented by the 2D acquisition result and the 3D acquisition result on an image display unit based on the assigned object coordinate dataset.

21. The method as claimed in claim 20, wherein the object is spatially or temporally jointly displayed.
